(19) United States
(12) Patent Application Publication
(10) Pub. No.: US 2023/0285441 A1
(43) Pub. Date: Sep. 14, 2023

(54) TREATMENT OF CORONAVIRUS INFECTIONS WITH AURANOFIN

(71) Applicant: GEORGIA STATE UNIVERSITY RESEARCH FOUNDATION, INC., Atlanta, GA (US)

(72) Inventor: Mukesh Kumar, Decatur, GA (US)

(21) Appl. No.: 17/996,318

(22) PCT Filed: Apr. 15, 2021
(86) PCT No.: PCT/US2021/027404

§ 371 Date: Nov. 28, 2022

Related U.S. Application Data
(60) Provisional application No. 63/010,336, filed on Apr. 15, 2020.

(51) Int. Cl. A61K 31/7135; A61K 45/06; A61P 31/14

(52) U.S. Cl. CPC A61K 31/7135; A61K 45/06; A61P 31/14

(57) ABSTRACT

The present disclosure provides methods for treating viral infections, in particular treating coronavirus infections in a subject with auranofin.

### CROSS-REFERENCE TO RELATED APPLICATIONS

This application claims the benefit of priority to U.S. Provisional Application No. 63/010,336, filed Apr. 15, 2020, the disclosure of which is incorporated herein by reference in its entirety.

### TECHNICAL FIELD

This disclosure relates to methods for treating viral infections, and more particularly to methods to treat coronavirus infections with auranofin.

### BACKGROUND

Coronaviruses are species of virus belonging to the subfamily Coronavirinae in the family Coronaviridae, in the order Nidovirales. Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large, bulbous surface projections creating an image reminiscent of a royal crown or of the solar corona. This morphology is created by the viral spike (S) peplomers, which are proteins that populate the surface of the virus and determine host tropism. Proteins that contribute to the overall structure of all coronaviruses are the spike (S), envelope (E), membrane (M) and nucleocapsid (N) proteins. In the specific case of the SARS coronavirus and SARS coronavirus 2, a defined receptor-binding domain on S mediates the attachment of the virus to its cellular receptor, angiotensin-converting enzyme 2 (ACE2). Some coronaviruses (specifically the members of Betacoronavirus subgroup A) also have a shorter spike-like protein called hemagglutinin esterase (HE).

In humans, coronaviruses cause respiratory tract infections that can range from mild to lethal. Mild illnesses include some cases of the common cold (which has other possible causes, predominantly rhinoviruses), while more lethal varieties can cause severe acute respiratory syndrome (SARS), Middle East respiratory syndrome (MERS), and coronavirus 2019 (COVID-2019). Symptoms in other species vary; in chickens, they cause an upper respiratory tract disease, while in cows and pigs they cause diarrhea.

Human coronaviruses vary significantly in risk factor. Some can kill more than 30% of those infected (such as MERS-CoV), and some are relatively harmless, such as the common cold. Coronaviruses cause colds with major symptoms, such as fever, and a sore throat from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). The human coronavirus discovered in 2003, SARS-CoV, which causes severe acute respiratory syndrome (SARS), has a unique pathogenesis because it causes both upper and lower respiratory tract infections.

Coronavirus disease 2019 (COVID-19) is an infectious disease caused by severe acute respiratory syndrome coronavirus 2 (SARS-CoV-2). The disease was first identified in December 2019 in Wuhan, the capital of China’s Hubei province, and has since spread globally, resulting in the ongoing coronavirus pandemic. Common symptoms include fever, cough, and shortness of breath. Other symptoms may include fatigue, muscle pain, diarrhea, sore throat, loss of smell, and abdominal pain. The time from exposure to onset of symptoms is typically around five days but may range from two to fourteen days. While the majority of cases result in mild symptoms, some progress to viral pneumonia and multi-organ failure. As of Apr. 8, 2021, more than 133 million cases have been reported across 210 countries and territories, resulting in over 2.89 million deaths.

Thus, there is a clear need for novel therapeutic methods for the treatment and/or prevention of coronavirus infections.

### SUMMARY

The present disclosure provides methods for treating coronavirus infections in a subject, in particular coronavirus disease 2019 (COVID-19), with auranofin, or a pharmaceutically acceptable salt or analog thereof. Auranofin was found to inhibit viral replication in coronavirus-infected cells in addition to providing an anti-inflammatory effect while showing no toxicity.

Thus in one aspect, a method for treating, inhibiting, decreasing, reducing, ameliorating and/or preventing a coronavirus infection and/or a method for treating, inhibiting, decreasing, reducing, ameliorating and/or preventing the disease and/or symptoms associated with said coronavirus infection in a subject in need thereof is provided comprising administering a therapeutically effective amount of auranofin, or a pharmaceutically acceptable salt or analog thereof. In some embodiments, the coronavirus causing the infection may be an alphacoronavirus, a betacoronavirus, a gammacoronavirus, or a deltacoronavirus. The coronavirus disease may be selected from a common cold, pneumonia, pneumonitis, bronchitis, severe acute respiratory syndrome (SARS), coronavirus disease 2019 (COVID19), Middle East respiratory syndrome (MERS), sinusitis, porcine diarrhea, porcine epidemic diarrhea, avian infections bronchitis, otitis and pharyngitis. In particular embodiments, the coronavirus disease comprises COVID-19. In some embodiments the coronavirus disease is caused by an infection with avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV.

Auranofin as used in the methods described herein may be administered as a pharmaceutical composition. Auranofin as used in the methods described herein may also be administered with one or more additional active agents, for example an antimicrobial agent, an anti-inflammatory agent, or an antiseptic agent.

A method is also provided for inhibiting replication of a coronavirus in a coronavirus-infected cell, the method comprising contacting the cell with auranofin, or a pharmaceutically acceptable salt or analog thereof.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts representative data which show that auranofin inhibits replication of SARS-CoV-2 in human cells. Huh7 cells were infected with SARS-COV-2 at a multiplicity of infection (MOI) of 1 for 2 hours and treated with 4 µM of auranofin or with 0.1% DMSO. Cell pellets and culture supernatants were collected at 24 and 48 hours after infection and viral RNA levels were measured by RT-PCR using primers and probe targeting the SARS-COV-2 N1 gene and the SARS-COV-2 N2 gene. The cellular RNA extracted from infected cells was quantified, normalized and viral RNA levels per µg of total cellular RNA were calculated. The results were identical for both set of primers showing dramatic reduction in viral RNA at both 24 and 48 hours. SARS-COV-2 infectivity titers were measured in cell culture supernatants at 48 hours after infection by plaque assay. Data represent the mean±SEM, representing two independent experiments conducted in duplicate, t-test p<0.001.

FIG. 2 depicts representative data that show a dose-dependent reduction in SARS-CoV-2 RNA in the auranofin-treated cells. The SARS-COV-2 infected Huh7 cells were treated with serial dilutions of auranofin (0.1 to 10 µM). Viral RNA in the cell pellets and culture supernatants were quantified by RT-PCR using primers and probe targeting the SARS-COV-2 N1. The data were plotted in graphs using non-linear regression model (GraphPad software). Auranofin inhibited virus replication in the infected cells at EC₅₀ of approximately 1.5 µM. The cytotoxic concentration of 50% was approximately 5.7 µM. Data represent two independent experiments conducted in duplicate.

FIG. 3 depicts representative data that shows that auranofin treatment dramatically reduced the expression of SARS-CoV-2-induced cytokines in human cells. mRNA levels of IL-6, IL-1β, TNFα and NF-kB were determined using qRT-PCR at 24 and 48 hours after infection. The fold change in infected cells compared to corresponding controls was calculated after normalizing to the GAPDH gene. Data represent the mean±SEM, representing two independent experiments conducted in duplicate.

Like reference symbols in the various drawings indicate like elements.

### DETAILED DESCRIPTION

The following description of the disclosure is provided as an enabling teaching of the invention in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which this invention belongs. The following definitions are provided for the full understanding of terms used in the specification.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra- articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject’s body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject’s body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC-(CH2)ₙ-COOH where n is 0-4, and the like, or using a different acid that produces the same counterion. Lists of additional suitable salts may be found, e.g., in Remington’s Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, the term “subject” or “host” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Auranofin

The methods of the present disclosure comprise administration of auranofin (1-Thio-β-D-glucopyranosatotriethylphosphine gold-2,3,4,6-tetraacetate) having the chemical structure:

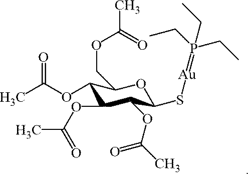

Auranofin is an antirheumatic agent that is traditionally used to treat rheumatoid arthritis, improving arthritis symptoms including painful or tender and swollen joints and morning stiffness. Auranofin is an FDA-approved drug that is widely available with minimal side effects.

### Methods of Treating Coronavirus Infections

The present disclosure provides methods for treating, inhibiting, decreasing, reducing, ameliorating and/or preventing a coronavirus infection in a subject in need thereof, the method comprising administering a therapeutically effective amount of auranofin, or a pharmaceutically acceptable salt or derivative thereof. In another aspect, the present disclosure provides methods for treating, inhibiting, decreasing, reducing, ameliorating and/or preventing the disease and/or symptoms associated with a coronavirus infection in a subject in need thereof, comprising administering a therapeutically effective amount of auranofin, or a pharmaceutically acceptable salt or derivative thereof. A “coronavirus infection” as used herein refers to an infection caused by or otherwise associated with growth of coronavirus in a subject, in the family Coronaviridae (subfamily Coronavirinae).

In one aspect, a method is provided for treating a coronavirus infection in a subject in need thereof, the method comprising administering a therapeutically effective amount of auranofin, or a pharmaceutically acceptable salt or derivative thereof.

In another aspect, a method is provided for treating a disease associated with a coronavirus infection in a subject in need thereof, the method comprising administering a therapeutically effective amount of auranofin, or a pharmaceutically acceptable salt or derivative thereof.

In another aspect, a method is provided for treating, inhibiting, decreasing, reducing, ameliorating and/or preventing one or more symptoms associated with a coronavirus infection in a subject in need thereof, the method comprising administering a therapeutically effective amount of auranofin, or a pharmaceutically acceptable salt or derivative thereof

Coronaviruses are species of virus belonging to the subfamily Coronavirinae in the family Coronaviridae, in the order Nidovirales. Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large, bulbous surface projections creating an image reminiscent of a royal crown or of the solar corona. This morphology is created by the viral spike (S) peplomers, which are proteins that populate the surface of the virus and determine host tropism. Proteins that contribute to the overall structure of all coronaviruses are the spike (S), envelope (E), membrane (M) and nucleocapsid (N) proteins. In the specific case of the SARS coronavirus and SARS coronavirus 2, a defined receptor-binding domain on S mediates the attachment of the virus to its cellular receptor, angiotensin-converting enzyme 2 (ACE2). Some coronaviruses (specifically the members of Betacoronavirus subgroup A) also have a shorter spike-like protein called hemagglutinin esterase (HE).

In one embodiment, the coronavirus infection is an infection of the upper and/or lower respiratory tract. The “upper respiratory tract” includes the mouth, nose, sinus, middle ear, throat, larynx, and trachea. The “lower respiratory tract” includes the bronchial tubes (bronchi) and the lungs (bronchi, bronchioles and alveoli), as well as the interstitial tissue of the lungs.

In another embodiment, the coronavirus infection is an infection of the gastrointestinal tract. The “gastrointestinal tract” may include any area of the canal from the mouth to the anus, including the mouth, esophagus, stomach, and intestines.

In yet another embodiments, the coronavirus infection is a renal infection.

It is understood and herein contemplated that the coronavirus infections disclosed herein can cause a pathological state associated with the coronavirus infection referred to herein as a “coronavirus disease.” In some embodiments, the coronavirus disease is selected from a common cold, pneumonia, pneumonitis, bronchitis, severe acute respiratory syndrome (SARS), coronavirus disease 2019 (COVID-2019), Middle East respiratory syndrome (MERS), sinusitis, porcine diarrhea, porcine epidemic diarrhea, avian infectious bronchitis, otitis and pharyngitis. In particular embodiments, the coronavirus disease is a common cold. In particular embodiments, the coronavirus disease is selected from SARS, COVID-19, and MERS. In a particular embodiment, the coronavirus disease is COVID-19. In another particular embodiment, the coronavirus disease is IBV, PorCoV HKU15, or PEDV.

Other indications associated with coronavirus infections are described in Gralinski & Baric, 2015, J. Pathol. 235:185-195 and Cavanagh, 2005, “Coronaviridae: a review of coronavirus and toroviruses”, Coronaviruses with Special Emphasis on First Insights Concerning SARS 1, ed. By A. Schmidt, M.H. Wolff and O. Weber, Birkhauser Verlag Baser, Switzerland, each of which is incorporated herein by reference in their entirety.

The coronavirus causing the infection may be selected from an alphacoronavirus, a betacoronavirus, a gammacoronavirus, or a deltacoronavirus.

Representative examples of alphacoronaviruses include, but are not limited to, a colacovirus (e.g., Bat coronavirus CDPHE15), a decacovirus (e.g., Bat coronavirus HKU10, Rhinolophus ferrumequinum alphacoronavirus Hub-2013), a duvinacovirus (e.g., Human coronavirus 229E), a luchacovirus (e.g., Lucheng Rn rat coronavirus), a minacovirus (e.g., Ferret coronavirus, Mink coronavirus 1), a minunacovirus (e.g., Miniopterus bat coronavirus 1, Miniopterus bat coronavirus HKU8), a myotacovirus (e.g., Myotis rickettii alphacoronavirus Sax-2011), a nyctacovirus (e.g., Nyctalus velutinus alphacoronavirus SC-2013), a pedacovirus (e.g., Porcine epidemic diarrhea virus (PEDV), Scotophilus bat coronavirus 512), a rhinacovirus (e.g., Rhinolophus bat coronavirus HKU2), a setracovirus (e.g., Human coronavirus NL63, NL63-related bat coronavirus strain BtKYNL63-9b), or a tegacovirus (e.g. Alphacoronavirus 1).

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang2013), a merbecovirus (e.g., Hedgehog coronavirus 1, Middle East respiratory syndrome-related coronavirus (MERS-CoV), Pipistrellus bat coronavirus HKU5, Tylonycteris bat coronavirus HKU4), a nobecovirus (e.g., Rousettus bat coronavirus GCCDC1, Rousettus bat coronavirus HKU9), or a sarbecovirus (e.g., severe acute respiratory syndrome coronavirus (SARS-CoV), severe acute respiratory syndrome coronavirus 2 (SARS-CoV-2).

Representative examples of gammacoronaviruses include, but are not limited to, a cegacovirus (e.g., Beluga whale coronavirus SQ1) or an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), or a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

In one embodiment, a method is provided for treating coronavirus disease 2019 (COVID-2019) comprising administering a therapeutically effective amount of auranofin, or a pharmaceutically acceptable salt or analog thereof.

In one embodiment, a method is provided for preventing coronavirus disease 2019 (COVID-2019) comprising administering a therapeutically effective amount of auranofin, or a pharmaceutically acceptable salt or analog thereof.

In another embodiment, a method is provided for inhibiting, decreasing, reducing, ameliorating and/or preventing one or more symptoms associated with coronavirus disease 2019 (COVID-2019) comprising administering a therapeutically effective amount of auranofin, or a pharmaceutically acceptable salt or analog thereof.

In another aspect, a method is provided for inhibiting replication of a coronavirus in a coronavirus-infected cell, the method comprising contacting the cell with a therapeutically effective amount of auranofin, or a pharmaceutically acceptable salt or analog thereof. In some embodiments, the coronavirus is SARS-CoV-2. In some embodiments, the cell is a human cell.

In another aspect, a method is provided for reducing viral load of a coronavirus in a coronavirus-infected cell, the method comprising contacting the cell with a therapeutically effective amount of auranofin, or a pharmaceutically acceptable salt or analog thereof. In some embodiments, the coronavirus is SARS-CoV-2. In some embodiments, the cell is a human cell.

In yet another aspect, a method is provided for reducing inflammation in a tissue of a subject infected with a coronavirus, the method comprising administering a therapeutically effective amount of auranofin, or a pharmaceutically acceptable salt or analog thereof. In some embodiments, the coronavirus is SARS-CoV-2. In some embodiments, the tissue is lung tissue.

In another aspect, a method is provided for reducing the expression of one or more inflammatory cytokines from a cell infected with a coronavirus, the method comprising contacting the cell with a therapeutically effective amount of auranofin, or a pharmaceutically acceptable salt or analog thereof. In some embodiments, the coronavirus is SARS-CoV-2. In some embodiments, the one or more inflammatory cytokines may be selected from IL-6, IL-1β, TNFα, and NF-κB. In some embodiments, the cell is a human cell.

### Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of an infection with a Mycobacterium.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington’s Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer’s solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinylpyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen- free water, isotonic saline, Ringer’s solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinylpyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer’s solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard- filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard- filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, auranofin as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents) and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some embodiments, auranofin as used in the methods described herein may administered in combination or alternation with one or more anticytokine or immunomodulatory agents, representative examples of which include, but are not limited to, tocilizumab, sarilumab, bevacizumab, fingolimod, imiquimod, and eculizumab.

In some embodiments, auranofin as used in the methods described herein may be administered in combination or alternation with an immunoglobulin therapy.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

SARS-COV-2 has recently emerged as a new public health threat. In the present example, it is demonstrated that the FDA-approved drug, auranofin, inhibits SARS-COV-2 replication in human cells at low micro molar concentration. Treatment of cells with auranofin resulted in 95% reduction in the viral RNA at 48 hours after infection. Auranofin treatment dramatically reduced the expression of SARS-COV-2-induced cytokines in human cells. These data indicate that auranofin is useful in limiting SARS-COV-2 infection and associated lung injury due to its antiviral, anti-inflammatory and anti-reactive oxygen species (ROS) properties.

Gold-based compounds have shown promising activity against a wide range of clinical conditions and microorganism infections. Auranofin, a gold-containing triethyl phosphine, is an FDA- approved drug for the treatment of rheumatoid arthritis since 1985 (Roder C, Thomson MJ. Auranofin: repurposing an old drug for a golden new age. Drugs in R&D. 2015;15(1):13-20). It has been investigated for potential therapeutic application in a number of other diseases including cancer, neurodegenerative disorders, HIV/AIDS, parasitic infections and bacterial infections (Harbut MB, Vilcheze C, Luo X, Hensler ME, Guo H, Yang B, Chatterjee AK, Nizet V, Jacobs WR, Jr., Schultz PG, Wang F. Auranofin exerts broad-spectrum bactericidal activities by targeting thiol-redox homeostasis. Proc Natl Acad Sci USA. 2015;112(14):4453-8. Epub 2015/04/02). Auranofin was approved by FDA for phase II clinical trials for cancer therapy (Hou GX, Liu PP, Zhang S, Yang M, Liao J, Yang J, Hu Y, Jiang WQ, Wen S, Huang P. Elimination of stem-like cancer cell side-population by auranofin through modulation of ROS and glycolysis. Cell death & disease. 2018;9(2):89; Oh BM, Lee SJ, Cho HJ, Park YS, Kim JT, Yoon SR, Lee SC, Lim JS, Kim BY, Choe YK, Lee HG. Cystatin SN inhibits auranofin-induced cell death by autophagic induction and ROS regulation via glutathione reductase activity in colorectal cancer. Cell death & disease. 2017;8(3):e2682; and Rigobello MP, Gandin V, Folda A, Rundlof AK, Fernandes AP, Bindoli A, Marzano C, Bjornstedt M. Treatment of human cancer cells with selenite or tellurite in combination with auranofin enhances cell death due to redox shift. Free radical biology & medicine. 2009;47(6):710-21). Oral auranofin was effective in rodent models of various parasitic infections (Leitsch D. Drug susceptibility testing in microaerophilic parasites: Cysteine strongly affects the effectivities of metronidazole and auranofin, a novel and promising antimicrobial. International journal for parasitology Drugs and drug resistance. 2017;7(3):321-7; and Capparelli EV, Bricker-Ford R, Rogers MJ, McKerrow JH, Reed SL. Phase I Clinical Trial Results of Auranofin, a Novel Antiparasitic Agent. Antimicrobial agents and chemotherapy. 2017;61(1). doi: 10.1128/AAC.01947-16). A preclinical study shows that auranofin significantly reduces HIV load in combination with antiretroviral therapy (Lewis MG, DaFonseca S, Chomont N, Palamara AT, Tardugno M, Mai A, Collins M, Wagner WL, Yalley-Ogunro J, Greenhouse J, Chirullo B, Norelli S, Garaci E, Savarino A. Gold drug auranofin restricts the viral reservoir in the monkey AIDS model and induces containment of viral load following ART suspension. Aids. 2011;25(11):1347-56). A clinical trial is ongoing to develop auranofin as a drug candidate to reduce the latent viral reservoir in patients with HIV infection utilizing the role of auranofin in affective redox-sensitive cell death pathways (Diaz RS, Shytaj IL, Giron LB, Obermaier B, Della Libera E, Jr., Galinskas J, Dias D, Hunter J, Janini M, Gosuen G, Ferreira PA, Sucupira MC, Maricato J, Fackler O, Lusic M, Savarino A, Group SW. Potential impact of the antirheumatic agent auranofin on proviral HIV-1 DNA in individuals under intensified antiretroviral therapy: Results from a randomised clinical trial. International journal of antimicrobial agents. 2019;54(5):592-600; and Chirullo B, Sgarbanti R, Limongi D, Shytaj IL, Alvarez D, Das B, Boe A, DaFonseca S, Chomont N, Liotta L, Petricoin EI, Norelli S, Pelosi E, Garaci E, Savarino A, Palamara AT. A candidate anti-HIV reservoir compound, auranofin, exerts a selective ‘anti-memory’ effect by exploiting the baseline oxidative status of lymphocytes. Cell death & disease. 2013;4:e944).

The mechanism of action of auranofin involves the inhibition of redox enzymes such as thioredoxin reductase, induction of endoplasmic reticulum (ER) stress and subsequent activation of the unfolded protein response (UPR) (May HC, Yu JJ, Guentzel MN, Chambers JP, Cap AP, Arulanandam BP. Repurposing Auranofin, Ebselen, and PX-12 as Antimicrobial Agents Targeting the Thioredoxin System. Frontiers in microbiology. 2018;9:336; Wiederhold NP, Patterson TF, Srinivasan A, Chaturvedi AK, Fothergill AW, Wormley FL, Ramasubramanian AK, Lopez-Ribot JL. Repurposing auranofin as an antifungal: In vitro activity against a variety of medically important fungi. Virulence. 2017;8(2):138-42; and Thangamani S, Mohammad H, Abushahba MF, Sobreira TJ, Seleem MN. Repurposing auranofin for the treatment of cutaneous staphylococcal infections. International journal of antimicrobial agents. 2016;47(3):195-201). Inhibition of these redox enzymes leads to cellular oxidative stress and intrinsic apoptosis (Lugea A, Gerloff A, Su HY, Xu Z, Go A, Hu C, French SW, Wilson JS, Apte MV, Waldron RT, Pandol SJ. The Combination of Alcohol and Cigarette Smoke Induces Endoplasmic Reticulum Stress and Cell Death in Pancreatic Acinar Cells. Gastroenterology. 2017;153(6):1674-86; and Hetz C. The unfolded protein response: controlling cell fate decisions under ER stress and beyond. Nature reviews Molecular cell biology. 2012;13(2):89-102). In addition, auranofin is an anti-inflammatory drug that reduces cytokines production and stimulate cell-mediated immunity (Walz DT, DiMartino MJ, Griswold DE, Intoccia AP, Flanagan TL. Biologic actions and pharmacokinetic studies of auranofin. The American journal of medicine. 1983;75(6A):90-108). It has been reported that auranofin interferes with the Interleukin 6 (IL-6) signaling by inhibiting phosphorylation of JAK1 and STAT3 (Han S, Kim K, Kim H, Kwon J, Lee YH, Lee CK, Song Y, Lee SJ, Ha N, Kim K. Auranofin inhibits overproduction of pro-inflammatory cytokines, cyclooxygenase expression and PGE2 production in macrophages. Archives of pharmacal research. 2008;31(1):67-74; and Kim NH, Lee MY, Park SJ, Choi JS, Oh MK, Kim IS. Auranofin blocks interleukin-6 signalling by inhibiting phosphorylation of JAK1 and STAT3. Immunology. 2007;122(4):607-14). The dual inhibition of inflammatory pathways and thiol redox enzymes by auranofin makes it an attractive candidate for cancer therapy and treating microbial infections.

Coronaviruses are a family of enveloped viruses with positive sense, single-stranded RNA genomes (Rothan HA, Byrareddy SN. The epidemiology and pathogenesis of coronavirus disease (COVID-19) outbreak. J Autoimmun. 2020:102433). SARS-CoV-2, the causative agent of COVID-19, is closely related to severe acute respiratory syndrome coronavirus (SARS-CoV-1) (Mehta P, McAuley DF, Brown M, Sanchez E, Tattersall RS, Manson JJ, Hlh Across Speciality Collaboration UK. COVID-19: consider cytokine storm syndromes and immunosuppression. Lancet. 2020;395(10229):1033-4). It is known that ER stress and UPR activation contribute significantly to the viral replication and pathogenesis during a coronavirus infection (Fung TS, Liu DX. Coronavirus infection, ER stress, apoptosis and innate immunity. Front Microbiol. 2014;5:296). Infection with SARS-COV-1 increases the expression of the ER protein folding chaperons GRP78, GRP94 and other ER stress related genes to maintain protein folding (Tang BS, Chan KH, Cheng VC, Woo PC, Lau SK, Lam CC, Chan TL, Wu AK, Hung IF, Leung SY, Yuen KY. Comparative host gene transcription by microarray analysis early after infection of the Huh7 cell line by severe acute respiratory syndrome coronavirus and human coronavirus 229E. Journal of virology. 2005;79(10):6180-93). Cells overexpressing the SARS-COV spike protein and other viral proteins exhibit high levels of UPR activation (Siu KL, Chan CP, Kok KH, Woo PC, Jin DY. Comparative analysis of the activation of unfolded protein response by spike proteins of severe acute respiratory syndrome coronavirus and human coronavirus HKU1. Cell & bioscience. 2014;4(1):3; and Sung SC, Chao CY, Jeng KS, Yang JY, Lai MM. The 8ab protein of SARS-CoV is a luminal ER membrane-associated protein and induces the activation of ATF6. Virology. 2009;387(2):402-13). Thus, inhibition of redox enzymes such as thioredoxin reductase and induction of ER stress by auranofin could significantly affect SARS-COV-2 protein synthesis (Rothan HA, Kumar M. Role of Endoplasmic Reticulum-Associated Proteins in Flavivirus Replication and Assembly Complexes. Pathogens. 2019;8(3)).

In addition, SARS-COV-2 infection causes acute inflammation and neutrophilia that leads to a cytokine storm with over expression of IL-6, TNF-alpha, monocyte chemoattractant protein (MCP-1) and reactive oxygen species (ROS). The severe COVID-19 illness represents a devastating inflammatory lung disorder due to cytokines storm that is associated with multiple organ dysfunction leading to high mortality (Sarzi-Puttini P, Giorgi V, Sirotti S, Marotto D, Ardizzone S, Rizzardini G, Antinori S, Galli M. COVID-19, cytokines and immunosuppression: what can we learn from severe acute respiratory syndrome? Clinical and experimental rheumatology. 2020;38(2):337-42). Taken together, these studies indicate that auranofin can mitigate SARS-COV-2 infection and associated lung damage due to its anti-viral, anti-inflammatory and anti-ROS properties.

### Results and Discussion

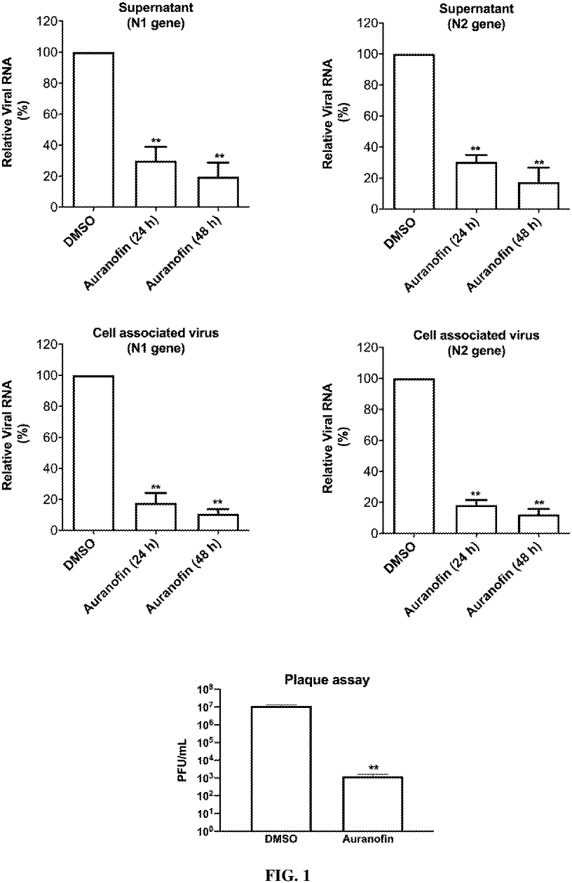

We investigated the anti-viral activity of auranofin against SARS-CoV-2 and its effect on virus-induced inflammation in human cells. We infected Huh7 cells with SARS-CoV-2 (USA-WA1/2020) at a multiplicity of infection (MOI) of 1 for 2 hours, followed by the addition of 4 µM of auranofin. DMSO (0.1%) was used as control (the solvent was used to prepare drug stock). We used Huh7 cells in this study as these cells are highly permissive for SARS-COV-2 infection. Cell culture supernatants and cell lysates were collected at 24 and 48 hours after infection. Virus RNA copies were measured by RT-PCR using two separate primers specific for the viral N1 gene and N2 gene (Rothan HA, Arora K, Natekar JP, Strate PG, Brinton MA, Kumar M. Z-DNA-Binding Protein 1 Is Critical for Controlling Virus Replication and Survival in West Nile Virus Encephalitis. Front Microbiol. 2019;10:2089; and Kumar M, Krause KK, Azouz F, Nakano E, Nerurkar VR. A guinea pig model of Zika virus infection. Virol J. 2017;14(1):75). As depicted in FIG. 1, treatment of cells with auranofin resulted in a 70% reduction in the viral RNA in the supernatant compared to the DMSO at 24 hours after infection. At 48 hours, there was an 85% reduction in the viral RNA in the supernatant compared to the DMSO. Similarly, the levels of intracellular viral RNA decreased by 85% at 24 hours and 95% at 48 hours in auranofin-treated cells compared to the DMSO-treated cells. Both set of primers showed nearly identical results. We next assayed virus titers in cell culture supernatants by plaque assay. Treatment with auranofin significantly reduced SARS-COV-2 infectivity titers in cell culture supernatants at 48 hours after infection (FIG. 1).

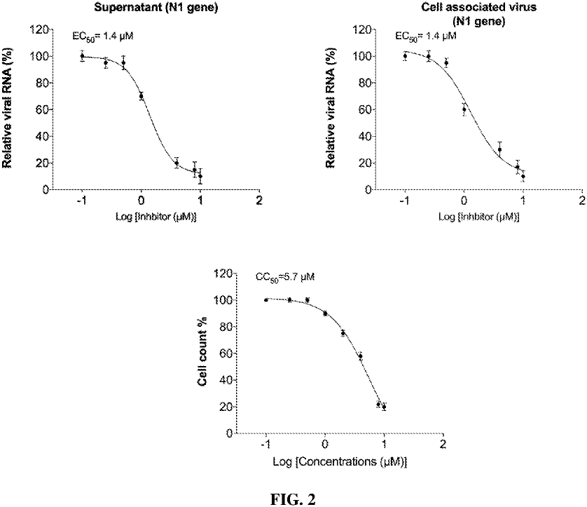

To determine the effective concentration of auranofin that inhibits 50% of viral replication (EC₅₀), we treated SARS-COV-2 infected Huh7 cells with serial dilutions of auranofin. Supernatants and cell lysates were collected at 48 hours after infection and viral RNA was quantified by RT-PCR. The data were plotted in graphs using non-linear regression model (GraphPad software). At 48 hours, there was a dose-dependent reduction in viral RNA levels in the auranofin-treated cells. FIG. 2 represents the EC₅₀ values of auranofin treatment against SARS-CoV-2 infected Huh7 cells. Auranofin inhibited virus replication in the infected cells at EC₅₀ of approximately 1.5 µM. It is important to note that in this example, we used 20 to 100-times more virus dose (MOI of 1) to infect the cells compared to the published reports on anti-viral activities of chloroquine, hydroxychloroquine, and remdesvir against SARS-COV-2 in vitro (Wang M, Cao R, Zhang L, Yang X, Liu J, Xu M, Shi Z, Hu Z, Zhong W, Xiao G. Remdesivir and chloroquine effectively inhibit the recently emerged novel coronavirus (2019-nCoV) in vitro. Cell research. 2020;30(3):269-71; and Liu J, Cao R, Xu M, Wang X, Zhang H, Hu H, Li Y, Hu Z, Zhong W, Wang M. Hydroxychloroquine, a less toxic derivative of chloroquine, is effective in inhibiting SARS-CoV-2 infection in vitro. Cell discovery. 2020;6:16).

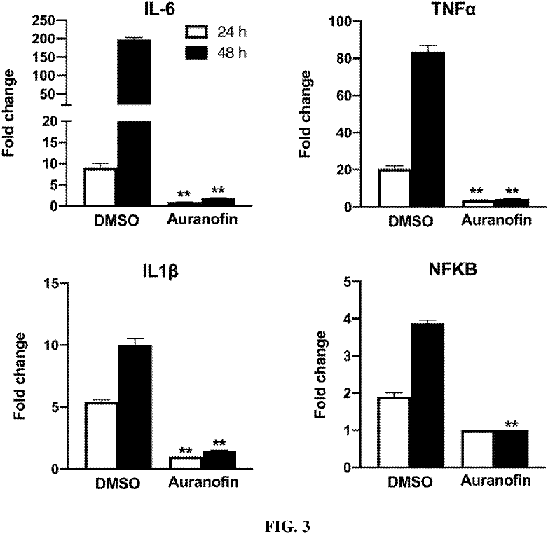

To assess the effect of auranofin on inflammatory response during SARS-COV-2 infection, we measured the levels of key cytokines in auranofin and DMSO-treated cells at 24 and 48 hours after infection (Natekar JP, Rothan HA, Arora K, Strate PG, Kumar M. Cellular microRNA-155 Regulates Virus-Induced Inflammatory Response and Protects against Lethal West Nile Virus Infection. Viruses. 2019;12(1)). SARS-COV-2 infection induces a strong up-regulation of IL-6, IL-1β, TNFα and NF-kB in Huh7 cells (FIG. 3). Treatment with auranofin dramatically reduced the expression of SARS-COV-2-induced cytokines in Huh7 cells. SARS-COV-2 infection resulted in a 200-fold increase in the mRNA expression of IL-6 at 48 hours after infection compared to corresponding mock-infected cells. In contrast, there was only a 2-fold increase in expression of IL-6 in auranofin-treated cells. TNF-α levels increased by 90-fold in the DMSO-treated cells at 48 hours after infection, but this increase was absent in the auranofin-treated cells. Similarly, no increase in the expression of IL-1β and NF-kB was observed in the auranofin-treated cells.

Taken together these results demonstrate that auranofin inhibits replication of SARS-COV-2 in human cells at low micro molar concentration. We also demonstrate that auranofin treatment resulted in significant reduction in the expression of cytokines induced by virus infection. These data indicate that auranofin could be a useful drug to limit SARS-CoV-2 infection and associated lung injury.

### Methods

### SARS-CoV-2 Infection and Drug Treatment:

In this study, we used a novel SARS-COV-2 (USA-WA1/2020) isolated from an oropharyngeal swab from a patient in Washington, USA (BEI NR-52281). Virus strain was amplified once in Vero E6 cells and had titers of 5 × 10⁶ plaque-forming units (PFU)/mL. Huh7 cells (human liver cell line) were grown in DMEM (Gibco) supplemented with 5% heat-inactivated fetal bovine serum. Cells were infected with SARS-COV-2 or PBS (Mock) at a multiplicity of infection (MOI) of 1 for 2 hours (Azouz F, Arora K, Krause K, Nerurkar VR, Kumar M. Integrated MicroRNA and mRNA Profiling in Zika Virus-Infected Neurons. Viruses. 2019;11(2); Kim JA, Seong RK, Kumar M, Shin OS. Favipiravir and Ribavirin Inhibit Replication of Asian and African Strains of Zika Virus in Different Cell Models. Viruses. 2018;10(2); and Krause K, Azouz F, Nakano E, Nerurkar VR, Kumar M. Deletion of Pregnancy Zone Protein and Murinoglobulin-1 Restricts the Pathogenesis of West Nile Virus Infection in Mice. Frontiers in microbiology. 2019;10:259). Cell were washed twice with PBS and media containing different concentrations of auranofin (0.1-10 µM, Sigma) or DMSO (0.1%, Sigma) was added to cells. Supernatants and cell lysates were harvested at 24 and 48 hours after infection. The cytotoxicity of auranofin in Huh7 cells was measured using trypan blue method as described previously (Varghese E, Busselberg D. Auranofin, an anti-rheumatic gold compound, modulates apoptosis by elevating the intracellular calcium concentration ([ca2+]I) in mcf-7 breast cancer cells. Cancers. 2014;6(4):2243-58). Briefly, Huh7 cells were treated with different concentrations of auranofin (0.1-10 µM) for 48 hours and percentage cell numbers were quantified using trypan blue.

### Viral RNA Quantification

Virus infectivity titers were measured in cell culture supernatants by plaque formation assay using Very cells as we described previously. Virus RNA levels were analyzed in the supernatant and cell lysates by quantitative reverse transcription-polymerase chain reaction (qRT-PCR). RNA from cell culture supernatants was extracted using a Viral RNA Mini Kit (Qiagen) and RNA from cell lysates was extracted using a RNeasy Mini Kit (Qiagen) as described previously. The cellular RNA extracted from infected cells was quantified, normalized and viral RNA levels per µg of total cellular RNA were calculated. qRT-PCR was used to measure viral RNA levels using primers and probes specific for the SARS-COV-2. Forward

(5′-GACCCCAAAATCAGCGAAAT-3′) (SEQ ID NO: 1)

, reverse

(5′-TCTGGTTACTGCCAGTTGAATCTG-3′) (SEQ ID NO: 2)

, probe,

(5′-FAM-ACCCCGCATTACGTTTGGTGGACC-BHQ1-3′) (SEQ ID
NO: 3)

targeting the SARS-COV-2 N1 gene and Forward

(5′-TTACAAACATTGGCCGCAAA-3′) (SEQ ID NO: 4)

, reverse

(5′-GCGCGACATTCCGAAGAA-3′) (SEQ ID NO: 5)

, probe,

(5′-FAM-ACAATTTGCCCCCAGCGCTTCAG-BHQ1-3′) (SEQ ID
NO: 6)

targeting the SARS-COV-2 N2 gene (Integrated DNA Technologies). Viral RNA copies were determined after comparison with a standard curve produced using serial 10-fold dilutions of SARS-COV-2 RNA.

### Cytokine Analysis:

For mRNA analysis of IL-6, IL-1β, TNFα and NF-kB, cDNA was prepared from RNA isolated from the cell lysates using a iScript™ cDNA Synthesis Kit (Bio-Rad, Hercules, CA, USA), and qRT-PCR was conducted as described previously. The fold change in infected cells compared to corresponding controls was calculated after normalizing to the GAPDH gene. The primer sequences used for qRT-PCR are listed in Table 1.

TABLE 1

Primer Sequences Used For qRT-PCR

| Gene (Accession No.) | Primer Sequence (5′-3′) |
|---|---|
| IL-1β (NM_000576) | |
| Forward | AGCACCTTCTTTCCCTTCATC (SEQ ID NO: 7) |
| Reverse | GGACCAGACATCACCAAGC (SEQ ID NO: 8) |
| IL-6 (NM_000600) | |
| Forward | CCAGGAGCCCAGCTATGAAC (SEQ ID NO: 9) |
| Reverse | CCCAGGGAGAAGGCAACTG (SEQ ID NO: 10) |
| NFKB (NM_003998) | |
| Forward | TCCTTCTTTGACTCATACA (SEQ ID NO: 11) |
| Reverse | TGCCTTCACATACATAACG (SEQ ID NO: 12) |
| Forward | CCTGCCCCAATCCCTTTATT (SEQ ID NO: 13) |
| Reverse | CCCTAAGCCCCCAATTCTCT (SEQ ID NO: 14) |

A number of embodiments of the invention have been described. Nevertheless, it will be understood that various modification may be made without departing from the spirit and scope of the invention. Accordingly, other embodiments are within the scope of the following claims.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method for treating a coronavirus infection or a coronavirus disease resulting from a coronavirus infection in a subject in need thereof, the method comprising administering a therapeutically effective amount of auranofin, or a pharmaceutically acceptable salt thereof.

2. (canceled)

3. The method of claim 1, wherein the coronavirus infection comprises an infection of the upper respiratory tract, an infection of the lower respiratory tract, an infection of the gastrointestinal tract, or a renal infection.

4-6. (canceled)

7. The method of claim 1, wherein the coronavirus disease is selected from a common cold, pneumonia, pneumonitis, bronchitis, severe acute respiratory syndrome (SARS), coronavirus disease 2019 (COVID-19), Middle East respiratory syndrome (MERS), sinusitis, porcine diarrhea, porcine epidemic diarrhea, avian infectious bronchitis, otitis, pharyngitis, IBV, PorCoV, HKU15, or PEDV.

8-11. (canceled)

12. The method of claim 1, wherein the coronavirus infection is the result of an alphacoronavirus, a betacoronavirus, a gamma coronavirus, or a deltacoronavirus.

13. The method of claim 12, wherein the alphacoronavirus is selected from a colacovirus,a decacovirus, a duvinacovirus, a luchacovirus, a minacovirus, a minunacovirus, a myotacovirus, a nyctacovirus, a pedacovirus, a rhinacovirus, a setracovirus, or a tegacovirus.

14. (canceled)

15. The method of claim 12, wherein the betacoronavirus is selected from an embecovirus 1, a hibecovirus, a nobecovirus, or a sarbecovirus.

16. (canceled)

17. The method of claim 12, wherein the gammacoronavirus is selected from a cegacovirus or an Igacovirus.

18. (canceled)

19. The method of claim 12, wherein the deltacoronavirus is selected from an andecovirus,a buldecovirus, a herdecovirus, or a moordecovirus.

20. The method of claim 1, wherein the subject is a human.

21. The method of 20, wherein the coronavirus infection is the result of a human coronavirus.

22. The method of claim 21, wherein the human coronavirus is selected from human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

23-25. (canceled)

26. The method of claim 1, wherein the subject is avian.

27. The method of claim 26, wherein the coronavirus is avian coronavirus (IBV).

28. The method of claims 1, wherein the subject is porcine.

29. The method of claim 28, wherein the coronavirus is porcine coronavirus HKU15 (PorCoV HKU15) or porcine epidemic diarrhea virus (PEDV).

30-32. (canceled)

33. The method of claim 1, wherein auranofin or its pharmaceutically acceptable salt is administered in combination with a pharmaceutically acceptable carrier as a pharmaceutical composition.

34. The method of claim 1, wherein auranofin or its pharmaceutically acceptable salt is administered in combination with one or more additional therapeutic agents selected from an antibiotic, an anti-viral agent, an anti-fungal agent, a steroid, a non-steroidal anti-inflammatory drug, or an antiseptic agent.

35. (canceled)

36. A method for inhibiting replication or for reducing viral load of a coronavirus in a coronavirus-infected cell, comprising contacting the cell with a therapeutically effective amount of auranofin, or a pharmaceutically acceptable salt or analog thereof.

37. (canceled)

38. A method for reducing expression of one or more inflammatory cytokines from a cell infected with a coronavirus, comprising contacting the cell with a therapeutically effective amount of auranofin, or a pharmaceutically acceptable salt or analog thereof.

39-53. (canceled)

54. The method of claim 36, wherein the cell is a human cell.


SEQUENCE LISTING (14 sequences)

SEQ ID NO: 1 - DNA, 20 bp, Artificial Sequence
  SARS-COV-2 N1 Forward Primer
gaccccaaaatcagcgaaat

SEQ ID NO: 2 - DNA, 24 bp, Artificial Sequence
  SARS-COV-2 N1 Reverse Primer
tctggttactgccagttgaatctg

SEQ ID NO: 3 - DNA, 24 bp, Artificial Sequence
  SARS-COV-2 N1 Probe
  misc_feature (1)..(1): 5' FAM substitution
  misc_feature (24)..(24): 3' BHQ1 substitution
accccgcattacgtttggtggacc

SEQ ID NO: 4 - DNA, 20 bp, Artificial Sequence
  SARS-COV-2 N2 Forward Primer
ttacaaacattggccgcaaa

SEQ ID NO: 5 - DNA, 18 bp, Artificial Sequence
  SARS-COV-2 N2 Reverse Primer
gcgcgacattccgaagaa

SEQ ID NO: 6 - DNA, 23 bp, Artificial Sequence
  SARS-COV-2 N2 Probe
  misc_feature (1)..(1): 5' FAM substitution
  misc_feature (23)..(23): 3' BHQ1 substitution
acaatttgcccccagcgcttcag

SEQ ID NO: 7 - DNA, 21 bp, Artificial Sequence
  IL-1beta Forward Primer
agcaccttctttcccttcatc

SEQ ID NO: 8 - DNA, 19 bp, Artificial Sequence
  IL-1beta Reverse Primer
ggaccagacatcaccaagc

SEQ ID NO: 9 - DNA, 20 bp, Artificial Sequence
  IL-6 Forward Primer
ccaggagcccagctatgaac

SEQ ID NO: 10 - DNA, 19 bp, Artificial Sequence
  IL-6 Reverse Primer
cccagggagaaggcaactg

SEQ ID NO: 11 - DNA, 19 bp, Artificial Sequence
  NFKB Forward Primer
tccttctttgactcataca

SEQ ID NO: 12 - DNA, 19 bp, Artificial Sequence
  NFKB Reverse Primer
tgccttcacatacataacg

SEQ ID NO: 13 - DNA, 20 bp, Artificial Sequence
  TNF Forward Primer
cctgccccaatccctttatt

SEQ ID NO: 14 - DNA, 20 bp, Artificial Sequence
  TNF Reverse Primer
ccctaagcccccaattctct